(19) United States
(12) Patent Application Publication
(10) Pub. No.: US 2011/0082517 A1
(43) Pub. Date: Apr. 7, 2011

(54) Device and Method for Activating the Natural Venous Muscle Pump using Electrical Stimulation Compounded by External Pressure

(75) Inventors: Yaakov B. Brezel, Jerusalem (IL); Shalom Lampert, Maalot (IL)

(73) Assignee: Empire Bio-Medical Devices Inc., Brooklyn, NY (US)

(21) Appl. No.: 12/965,883

(22) Filed: Dec. 12, 2010

Related U.S. Application Data
(63) Continuation-in-part of application No. PCT/IL2009/000584, filed on Jun. 14, 2009.
(60) Provisional application No. 61/060,853, filed on Jun. 12, 2008.

(51) Int. Cl. A61N 1/00

(52) U.S. Cl. 607/48

(57) ABSTRACT

A device and method for promoting a localized increase in a flow of blood through veins in a conical limb segment of a user, the device including: (a) a compression unit, adapted to at least partially envelope the conical limb segment, and (b) an electrical stimulation unit including: (i) a plurality of electrodes, each adapted to operatively contact the limb segment; (ii) a signal generator, operatively connected to at least one of the electrodes, and adapted to electrically connect to a power supply, whereby, when the electrodes are disposed on the limb segment, the signal generator provides a series of electrical impulses to the limb segment via the electrodes, to contract at least one muscle in the limb segment in at least one region of contraction, to effect the localized increase in the flow of blood, and (iii) a control unit, associated with the signal generator, and configured to control the signal generator to produce the series of electrical stimulation impulses, the electrodes physically attached to the compression unit and at least partially disposed thereunder, the compression unit having an inside face adapted to deliver, to a surface of the limb segment, a constant, superatmospheric pressure of at least 5 mmHg.

### CROSS-REFERENCE TO RELATED APPLICATIONS

This patent application is a continuation-in-part of PCT/IL2009/000584 filed on Jun. 14, 2009 and published as WO/2009/150652, and claims priority to U.S. Provisional Patent Application 61/060,853 filed on Jun. 12, 2008, which are all hereby incorporated in their entirety by reference.

### FIELD AND BACKGROUND OF THE INVENTION

The present invention relates to a method and device for promoting a localized increase in a venous flow of blood, and more particularly, to a non-invasive method and device for promoting a localized increase in the venous flow of blood through the extremities of a patient, utilizing electrical stimulation compounded by a constant external pressure.

The localized increase in the flow of blood effected by the device and method of the present invention may be important for a wide variety of medical applications, including alleviating chronic venous insufficiency (CVI), inhibiting deep vein thrombosis (DVT), treating phlebitis, decreasing the amount of retained water in the lower limbs, improving blood and lymph circulation, thereby alleviating pain, and speeding up healing, particularly in the case of venous ulcers and the like.

The use of electrical stimulation to effect such a localized increase in the venous flow of blood is known. Examples of such electrical stimulation devices are disclosed in U.S. Pat. No. 5,674,262 and U.S. Patent Publication No. 20070270917, both of which are incorporated by reference for all purposes as if fully set forth herein.

It is believed that there is a need for further improvements in non-invasive methods and devices for promoting a localized increase in the venous flow of blood through the extremities, and the subject matter of the present disclosure and claims is aimed at fulfilling this need.

### SUMMARY OF THE INVENTION

According to the teachings of the present invention there are provided a non-invasive device and method for promoting a localized increase in a flow of blood through veins in a conical limb segment of a body, substantially as described herein, the device and method including any feature described, either individually or in combination with any feature, in any configuration.

According to another aspect of the present invention there is provided a non-invasive neuromuscular device for promoting a localized increase in a flow of blood through veins in a conical limb segment of a user by means of muscular contraction within the limb segmented compounded by mechanical compression, the device including: (a) a compression unit, adapted to at least partially envelope the conical limb segment, and (b) an electrical stimulation unit including: (i) a plurality of electrodes including at least a first electrode and a second electrode, each of the first and second electrodes adapted to operatively contact the limb segment; (ii) at least one signal generator, each generator operatively connected to at least one of the electrodes, the at least one signal generator adapted to electrically connect to a power supply, whereby, when the plurality of electrodes are disposed on the limb segment, the at least one signal generator provides a series of electrical impulses to the limb segment via the plurality of electrodes to contract at least one skeletal muscle in the limb segment in at least one region of contraction, to effect the localized increase in the flow of blood through the veins, and (iii) a control unit, associated with the signal generator, the control unit designed and configured to control the signal generator to produce the series of electrical stimulation impulses, the electrodes physically attached to the compression unit and at least partially disposed thereunder, wherein the conical limb segment is selected from the group consisting of an upper arm limb segment, a lower arm limb segment, an upper leg limb segment and a lower leg limb segment, the compression unit having an inside face adapted to deliver, to a surface of the limb segment, a superatmospheric pressure that is substantially constant over time, the pressure equaling at least 5 mmHg, at least 8 mmHg, at least 10 mmHg, at least 12 mmHg, or at least 16 mmHg.

According to yet another aspect of the present invention there is provided a non-invasive device for promoting a localized increase in a flow of blood through veins in a conical limb segment of a user, the device including: (a) a compression unit, adapted to envelope a majority of a circumference of the conical limb segment, and (b) an electrical stimulation unit including: (i) a plurality of electrodes including at least a first electrode and a second electrode, each of the first and second electrodes adapted to operatively contact the limb segment; (ii) at least one signal generator, each generator operatively connected to at least one of the electrodes, the at least one signal generator adapted to electrically connect to a power supply, the at least one signal generator providing a series of electrical impulses to the limb segment via the plurality of electrodes to contract at least one muscle in the limb segment in at least one region of contraction, to effect the localized increase in the flow of blood through the veins, and (iii) a control unit, associated with the signal generator, the control unit designed and configured to control the signal generator to produce the series of electrical stimulation impulses, the electrodes at least partially disposed under the compression unit, wherein the conical limb segment is selected from the group consisting of an upper arm limb segment, a lower arm limb segment, an upper leg limb segment and a lower leg limb segment, the compression unit having an inside face adapted to deliver, to a surface of the limb segment, a superatmospheric pressure that is substantially constant over time, the pressure equaling at least 5 mmHg, and wherein, when the compression unit is in a relaxed state, the first and second electrodes protrude from the inside face by less than 0.6 mm, less than 0.4 mm or less than 0.25 mm.

According to yet another aspect of the present invention there is provided a non-invasive device for promoting a localized increase in a flow of blood through veins in a limb segment of a body, the device including: (a) a compression unit, adapted to at least partially envelope the limb segment, and (b) an electrical stimulation unit including: (i) a plurality of electrodes including at least a first electrode and a second electrode, each of the electrodes adapted to operatively contact the limb segment of the body; (ii) at least one signal generator, each generator operatively connected to at least one of the electrodes, the at least one signal generator adapted to electrically connect to a power supply, the at least one signal generator providing a series of electrical impulses to the limb segment via the plurality of electrodes to contract at least one muscle in the limb segment in at least one region of contraction, to effect the localized increase in the flow of blood through the veins, and (iii) a control unit, associated with the signal generator, the control unit designed and configured to control the signal generator to produce the series of electrical stimulation impulses, at least one of the electrodes physically associated with the compression unit and at least partially disposed thereunder, the compression unit adapted to deliver a pressure to a surface of the limb segment.

According to yet another aspect of the present invention there is provided a non-invasive method for promoting a localized increase in a flow of blood through veins in a conical limb segment of a body, the method including the steps of: (a) providing a device including: (i) a compression unit, adapted to at least partially envelope the limb segment, and (ii) an electrical stimulation unit including: (A) a plurality of electrodes including at least a first electrode and a second electrode, each of the electrodes adapted to operatively contact the limb segment; (B) at least one signal generator, each generator operatively connected to at least one of the electrodes, the signal generator adapted to electrically connect to a power supply, and (C) a control unit, associated with the signal generator, the control unit designed and configured to control the signal generator to produce a series of electrical stimulation impulses, (b) positioning the plurality of electrodes on the limb segment; (c) controlling the series of electrical stimulation impulses to induce a plurality of contractions of muscular tissue in at least one region of contraction on the limb segment; (d) positioning the compression unit on the limb segment, to at least partially cover the at least one region of contraction, and (e) exerting, on a surface of the limb segment, by means of the compression unit, a superatmospheric pressure that is substantially constant over time, the pressure equaling at least 5 mmHg, at least 8 mmHg, at least 12 mmHg, or at least 16 mmHg, wherein the conical limb segment is selected from the group consisting of an upper arm limb segment, a lower arm limb segment, an upper leg limb segment and a lower leg limb segment, and wherein steps (c) and (e) promote the localized increase in the flow of blood through the veins.

According to further features in the described preferred embodiments, the compression unit is selected from the group consisting of at least one compression bandage and at least one compression garment.

According to further features in the described preferred embodiments, the compression unit is adapted to at least partially envelope a lower leg of the user.

According to further features in the described preferred embodiments, the compression unit is further adapted, and disposed with respect to the electrodes, whereby the superatmospheric pressure is delivered to a calf of the lower leg, in a substantially identical location as a contraction of the skeletal muscle.

According to further features in the described preferred embodiments, the compression unit is adapted to at least partially envelope a gastrocnemius muscle of the lower leg of the user.

According to further features in the described preferred embodiments, the compression unit is adapted to deliver, to the surface of the limb segment, a maximal pressure below 80 mmHg, below 60 mmHg, below 50 mmHg, below 40 mmHg, or below 35 mmHg.

According to further features in the described preferred embodiments, the inside face is adapted to contact the conical limb segment, the face having at least one receiving area adapted to receive the first and second electrodes.

According to still further features in the described preferred embodiments, the first and second electrodes protrude from the inside face by less than 0.6 mm, less than 0.4 mm or less than 0.25 mm.

According to still further features in the described preferred embodiments, when the compression garment is in a relaxed state, the first and second electrodes protrude from the inside face by less than 0.6 mm, less than 0.4 mm or less than 0.25 mm.

According to still further features in the described preferred embodiments, when the compression garment is in a relaxed state, the first and second electrodes are disposed within the garment, whereby a contact surface of the first and second electrodes is substantially flush with the inside face.

According to still further features in the described preferred embodiments, the compression unit is an elastic compression unit.

According to still further features in the described preferred embodiments, the compression unit is still further adapted to envelope the limb segment to deliver the pressure to at least one region of contraction on the limb segment.

According to still further features in the described preferred embodiments, the compression unit is further adapted to envelop at least 60%, at least 80%, or at least 90% of a circumference of the limb segment.

According to still further features in the described preferred embodiments, the compression unit is further adapted to envelop substantially an entire circumference of the limb segment.

According to still further features in the described preferred embodiments, the inside face has a first zone adapted to deliver a first pressure against a first portion of the surface of the limb segment and a second zone adapted to deliver a second pressure against the limb segment, the first pressure exceeding the second pressure.

According to still further features in the described preferred embodiments, the first pressure exceeds the second pressure by at least 5 mm Hg, at least 10 mmHg or by at least 15 mmHg.

According to still further features in the described preferred embodiments, at least one of the first and second electrodes is disposed within the second zone.

According to still further features in the described preferred embodiments, the absolute pressure of the second pressure is less than 12 mmHg, less than 8 mmHg or less than 4 mmHg.

According to still further features in the described preferred embodiments, the limb segment is on a leg of the body, at least partially below a knee.

According to still further features in the described preferred embodiments, the lower leg limb segment includes a calf at least a portion of a gastrocnemius muscle of the leg.

According to still further features in the described preferred embodiments, the plurality of electrodes further includes at least a third electrode or at least a third electrode and a fourth electrode.

According to still further features in the described preferred embodiments, the electrical stimulation unit may be adapted to deliver a voltage of at least 40V, at least 50 Volts, at least 60 Volts, or at least 70 Volts. Typically, the voltage is within a range of 60V-100V.

According to still further features in the described preferred embodiments, the device further includes: (c) a switching mechanism designed and configured for switching electrical connections between the signal generator and at least each of the first, second, and third electrodes, according to a pre-determined sequence, to provide a first voltage differential between the first electrode and the second electrode, a second voltage differential between the second electrode and the third electrode, and a third voltage differential between the third electrode and another electrode of the plurality of electrodes.

According to still further features in the described preferred embodiments, at least two of the plurality of electrodes are disposed proximate to a first end of the compression bandage or the compression garment, and at least another two of the plurality of electrodes are disposed proximate to an opposite end of the compression bandage or the compression garment.

According to still further features in the described preferred embodiments, at least two of the plurality of electrodes are disposed proximate to a first end of the compression bandage or the compression garment, and at least another one of the plurality of electrodes is disposed proximate to an opposite end of the compression bandage or the compression garment.

According to still further features in the described preferred embodiments, the switching mechanism is responsive to the control unit.

According to still further features in the described preferred embodiments, the pre-determined sequence is a repeating sequence.

According to still further features in the described preferred embodiments, the control unit configured whereby the repeating sequence has a frequency of 1-30 periods per minute, or 5-20 periods per minute.

According to still further features in the described preferred embodiments, the thickness of the compression garment above the receiving area is less than a thickness of the compression garment outside of the receiving area.

According to still further features in the described preferred embodiments, the thickness of the compression garment above the second zone is less than a thickness of the compression garment above the first zone by at least 2 mm, at least 1 mm, or at least 0.5 mm.

According to still further features in the described preferred embodiments, a first material of the compression garment, disposed above the first zone, is selected to exert a higher compressive force with respect to a second material of the compression garment, disposed above the second zone.

According to still further features in the described preferred embodiments, the compression unit includes a multiple layer compression bandage.

According to still further features in the described preferred embodiments, a maximal pressure of the compression unit is below 80 mmHg, below 60 mmHg, below 50 mmHg, below 40 mmHg, or below 35 mmHg.

According to still further features in the described preferred embodiments, the contractions exert a pressure on at least one deep vein in the limb segment to augment the localized increase in the flow of blood through the limb segment.

According to still further features in the described preferred embodiments, a duration of each of the electrical impulses is within a range of 80-1200 microseconds, or within a range of 100-600 microseconds.

According to still further features in the described preferred embodiments, the compression unit is positioned on the limb segment, to completely cover the at least one region of contraction.

According to still further features in the described preferred embodiments, the compression unit is positioned on the limb segment, to completely cover a center of the at least one region of contraction.

According to still further features in the described preferred embodiments, the compression unit is positioned on the limb segment, to completely cover a central area of the at least one region of contraction, the central area being a circular area covering an area of at least 6 cm² around a center of contraction on the limb segment.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are used to designate like elements.

In the drawings:

FIG. 1 is a block diagram showing the components of a prior art stimulation device that may form a part of the device of the present invention;

FIGS. 2A-2D provide schematic illustrations of a section of a lower leg, to which are affixed electrodes, according to the prior art, along with a schematic representation of the contraction sequence provided by the control unit;

FIG. 3 is a schematic side view of an exemplary compression device that may form a portion of the device of the present invention;

FIG. 4 provides a schematic, cross-sectional view of a compressive sock that may be donned directly on the limb segment, in accordance with the present invention;

FIG. 5 shows a schematic side view of the inventive compressive sock of FIG. 4, disposed on a leg of the user;

FIG. 6 is a schematic perspective view of a portion of an inside face of a device of the present invention, in which surface electrodes are embedded;

FIG. 7A is a schematic, cross-sectional view of a portion of the inside face of FIG. 6, the compression garment or bandage having a recess or void volume behind the electrodes, and

FIG. 7B is a schematic, cross-sectional view of a portion of the inside face of FIG. 6, in which the recess of FIG. 7A is at least partially filled with a filler material, according to another embodiment of the present invention.

### DESCRIPTION OF THE PREFERRED EMBODIMENTS

One aspect of the present invention is a method and device for externally promoting a localized increase in a venous flow of blood in an extremity or peripheral region of the body, typically in a leg of the patient, by activating the natural venous muscle pump using electrical stimulation compounded by external pressure.

This sequence of repeated contractual movement of muscular tissue may be reversed, such that the flow of blood to a given area may be reduced.

Such methods and devices may be used in a substantially painless, external, and non-invasive manner. Moreover, the device of the present invention is simple, easy to adjust, readily adaptable to the needs of a specific patient, and can be operated by the typical patient.

The principles and operation of the device and process of the present invention may be better understood with reference to the drawings and the accompanying description. Before explaining at least one embodiment of the invention in detail, it is to be understood that the invention is not limited in its application to the details of construction and the arrangement of the components set forth in the following description or illustrated in the drawing. The invention is capable of other embodiments or of being practiced or carried out in various ways. Also, it is to be understood that the phraseology and terminology employed herein is for the purpose of description and should not be regarded as limiting.

As used herein, the term “voltage differential” refers to an absolute difference between two distinct voltage values. Such a voltage differential is at least 5 Volts, and more typically, at least 10 Volts.

As used herein in the specification and in the claims section that follows, the term “muscle contraction” and the like, with respect to a limb segment, refers to an instrumentally induced contraction of at least one muscle, the contraction causing a transitory constriction of a vein within the limb segment.

As used herein in the specification and in the claims section that follows, the term “point of contraction”, “contraction point”, and the like, with respect to at least one muscle on a limb, refers to the center of the muscle contraction along the length of that limb.

As used herein in the specification and in the claims section that follows, the term “center of contraction” and the like, with respect to a muscle on a limb segment, refers to an area or region surrounding the exact center of a muscle contraction causing a transitory constriction in a vein within the limb segment. This area or region is typically circular, and the size of the area or region may be less than 6 cm², typically, less than 4 cm², and more typically, less than 2 cm².

As used herein in the specification and in the claims section that follows, the term “conical limb segment” and the like, refers to any one of four different limb segments of the body: (1) upper arm, between the shoulder and elbow; (2) lower arm, between the elbow and wrist; (3) upper leg, between the hip and knee; and (4) lower leg, between the knee and ankle. As understood by those of skill in the art, the hand and foot sections of the limbs are not included in the term “conical limb segment”, and the term is meant to expressly excluded the hand and foot sections of the limbs.

The specification and claims refer to a superatmospheric pressure delivered to a surface of a limb segment by a compression unit such as a compression garment or compression bandage. It is manifest that the exact pressure delivered by a particular bandage or garment may vary somewhat from limb segment to limb segment. For the sake of clarity, the term “superatmospheric pressure delivered to a surface of a limb segment”, and the like, may be defined in terms of the superatmospheric pressure delivered to a surface of an average limb segment. In the case of the lower leg, the average limb segment has an ankle circumference within a range of about 18 to about 24 cm.

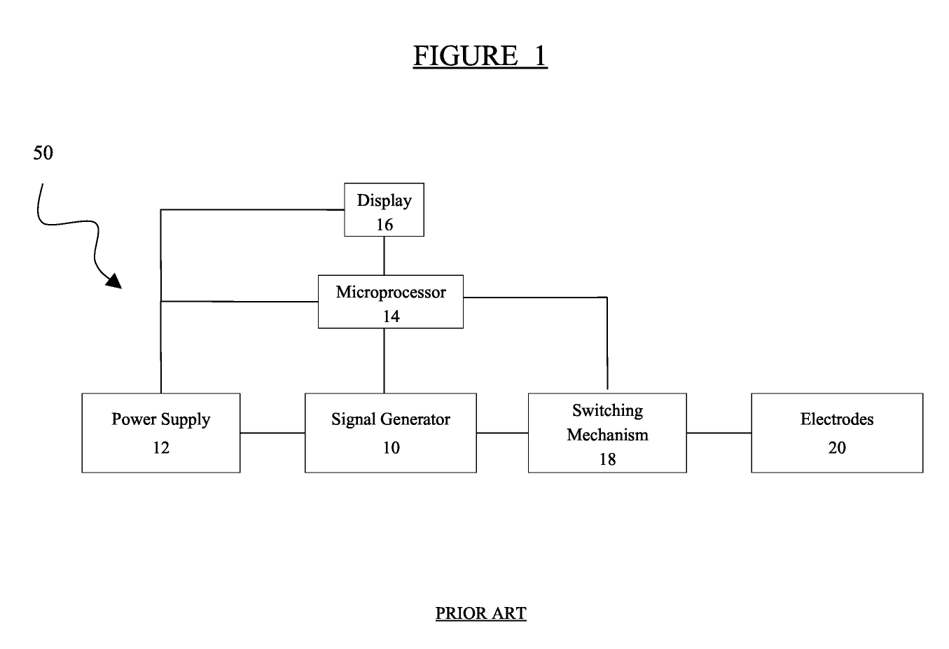

Referring now to the drawings, FIG. 1 is a block diagram showing the components of a typical stimulation device 50 that may form a part of the device of the present invention. At least one signal generator such as signal generator 10 may be operatively connected to a power supply 12. Also connected to power supply 12, are control unit or microprocessor 14 and display 16. Signal generator 10 may also be integral with microprocessor 14. Signal generator 10 may also be operatively connected to at least two electrodes such as electrodes 20, optionally via a switching mechanism 18. Control unit 14 may control signal generator 10 to produce a series of electrical stimulation impulses. These impulses are delivered to electrodes 20 positioned on a limb segment of the patient. When three or more electrodes are used, switching mechanism 18 may determine to which pair of electrodes the stimulation impulses will be delivered. Switching mechanism 18 may also be configured as a distributing mechanism adapted to simultaneously distribute a positive or negative signal to two or more electrodes.

Display 16, which is responsive to control unit 14, may be advantageously configured to display information such as signal frequency, pulse width, period, and voltage.

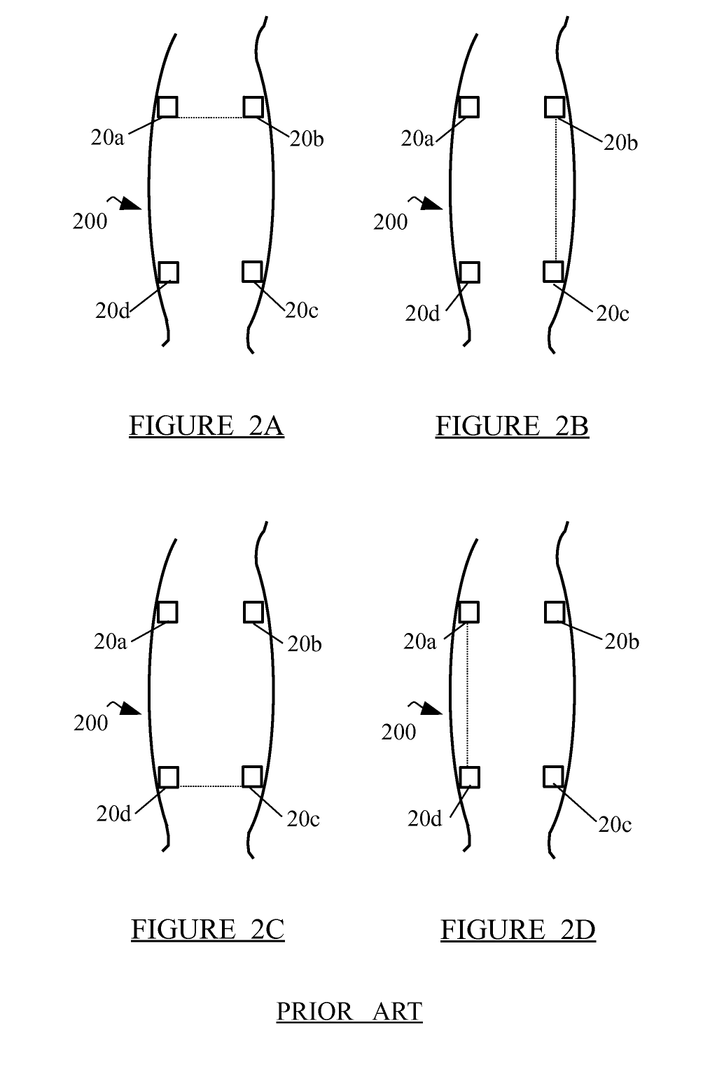

Examples of such electrical stimulation devices are disclosed in U.S. Pat. No. 5,674,262 and U.S. Patent Publication No. 20070270917. In U.S. Patent Publication No. 20070270917, FIG. 2A provides a schematic illustration of a section of a conical limb segment such as lower leg 200, to which are affixed electrodes 20a-d. A first pair of electrodes 20a-b is affixed at an upper end of lower leg 200 and a second pair of electrodes 20c-d is affixed at the opposite end of lower leg 200.

By applying a suitable voltage differential and current to electrodes 20a-d, muscular tissue in lower leg 200 contracts, thereby impinging upon the local blood vessels. U.S. Patent Publication No. 20070270917 discloses that with the proper electrical impulses and contraction positioning (constriction points), and timing sequence, the device can be utilized to appreciably, measurably, and repeatably enhance the flow of blood through the limb segment.

The timing sequence will now be described, by way of example, with reference to FIG. 1 and FIGS. 2A-2D. In step (I), shown schematically in FIG. 2A, switching mechanism 18 delivers a voltage differential from signal generator 10 (shown in FIG. 1) to a first pair of electrodes 20a-b disposed at an upper end of lower leg 200. As a result, a muscular contraction 40 is effected between electrodes 20a-b. In step (II), shown schematically in FIG. 2B, switching mechanism 18 delivers a voltage differential from signal generator 10 to an electrode from first pair of electrodes 20a-b and to an electrode from a second pair of electrodes 20c-d disposed at a lower end of lower leg 200. The resulting muscular contraction is substantially a longitudinal muscular contraction 42 along the length of lower leg 200.

In step (III), shown schematically in FIG. 2C, switching mechanism 18 delivers a voltage differential from signal generator 10 to second pair of electrodes 20c-d. Consequently, a muscular contraction 44 is effected between electrodes 20c-d at or towards a lower end of lower leg 200.

Step (IV), shown schematically in FIG. 2D, completes the cycle: switching mechanism 18 delivers a voltage differential from signal generator 10 to an electrode from first pair of electrodes 20a-b and to an electrode from second pair of electrodes 20c-d, so as to effect a substantially longitudinal muscular contraction 46 along the length of lower leg 200.

We have discovered that a localized increase in a venous flow of blood in an extremity or in a peripheral region of the body can be achieved by compounding electrical stimulation of the natural venous muscle pump with external pressure, more specifically, a compression unit such as a compression garment or compression bandage. While compression garments may provide a modest benefit in augmenting superficial venous flow, such compression garments are generally known to be ineffective in improving the flow of blood through the underlying deep veins.

At least a portion of at least one electrode of the electrical stimulation device may be disposed underneath the compression unit, between the compression unit and the calf of the leg being stimulated. The electrode may be completely disposed underneath the compression unit.

Preferably, the compression garment or bandage may be adapted and disposed, with respect to the electrodes, such that pressure is delivered to the calf in substantially the same location as the contraction of the superficial muscle takes place. If, by way of example, the electrical stimulation contracts the gastrocnemius muscle, the compression garment delivers pressure to the calf to that gastrocnemius muscle. Preferably, the compression unit delivers pressure to the calf at the central point or region of the gastrocnemius muscle contraction.

We have further discovered that the use of external pressure on a conical limb segment, to augment the effect of the electrical stimulation of the natural venous muscle pump, may, under particular conditions, achieve substantially no additional localized increase in a venous flow of blood, with respect to the electrical stimulation alone. More surprisingly, the use of external pressure to compound the electrical stimulation of the natural venous muscle pump may, in some circumstances, actually impair the performance of the electrical stimulation device, such that the venous flow of blood, with respect to the electrical stimulation alone, is actually reduced. This phenomenon may occur when the maximal pressure exerted by the compression unit is above 60 mmHg, above 50 mmHg, or above 40 mmHg. In some cases, for example, in patients having weak muscles, this phenomenon may occur when the maximal pressure exerted by the compression unit is above 35 mmHg. Below these maximal pressure levels, however, the use of external pressure to compound the electrical stimulation of the natural venous muscle pump may be particularly efficacious.

Without wishing to be bound by theory, we believe that at relatively high compressive pressures, the pressure delivered to the walls of the veins is sufficiently high to constrict the flow therethrough, such that the pressure is a deleterious influence on the action of the electrical stimulation on the venous muscle pump. This problem may be significantly more pronounced in patients having a weak natural venous muscle pump.

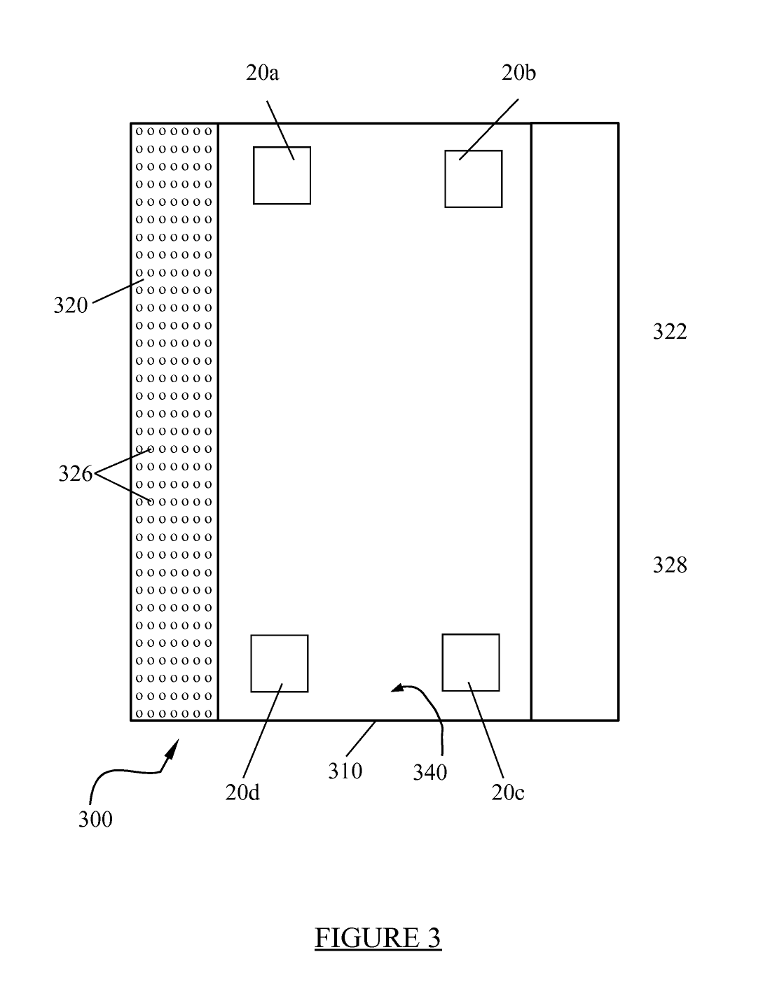

FIG. 3 is a schematic side view of an exemplary compression device 300 that may form a portion of the device of the present invention. Compression device 300 includes a flexible sheet 310 that may be generally rectangular. On a first, inside face 340 of flexible sheet 310 are disposed electrodes, such as electrodes 20a-d, which may be secured to flexible sheet 310 by various fastening elements and arrangements known in the art.

The flexible sheet is designed and dimensioned to at least partially envelope a limb segment of the user. By way of example, flexible sheet 310 is designed and dimensioned to substantially completely envelope a limb segment such as the lower leg of the user.

Compression device 300 may be wrapped around the limb segment and tightly secured to the limb segment using fastening elements and arrangements known in the art, including various complementary fastening arrangements, depending on the desired pressure and other considerations. In FIG. 3, flexible sheet 310 is equipped with an exemplary loop and hook fastening arrangement having a first region 320 containing loops 326 and a second region 322 containing hooks 328. Typically, second region 322 is disposed on the first face of flexible sheet 310, and first region 320 is disposed on the opposite face (denoted by dashed lines) of flexible sheet 310, such that first and second regions 320, 322 overlap and contact each other, thereby fastening loops 326 and hooks 328.

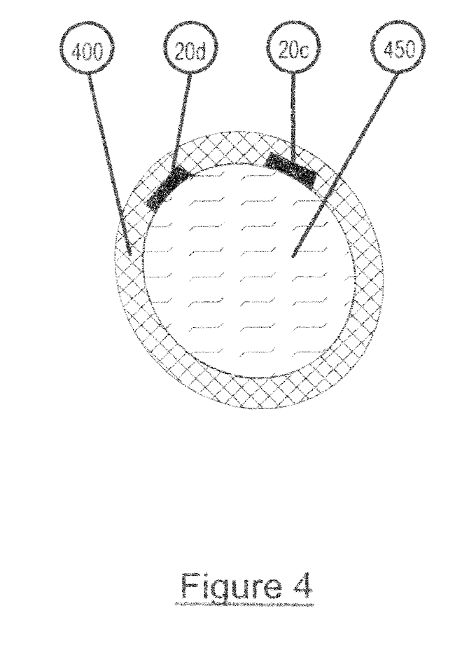

Additional pressure on the limb segment may be developed by donning a compressive garment or sock on top of compression device 300. FIG. 4 shows a schematic cross-sectional view of such a compressive sock 400, disposed on a leg 450 of the user.

Alternatively, and as shown in FIG. 4, compressive sock 400 may be donned directly on the limb segment, with no additional compression device disposed therebetween. Compressive sock 400 may cover, or at least partially cover, the electrodes such as electrodes 20c and 20d.

Various types of constant support and constant compression bandages are known in the art, and may be used as part of the device and method of the present invention. Light support bandages, including various crepe-type arrangements, have seen use in preventing edema formation.

More typically, light compression bandages may be used for this purpose. Light compression bandages may provide and maintain low levels of pressure, up to 20 mmHg on an ankle of average dimensions.

Moderate compression bandages may be used to apply compression of about 30 mmHg. High compression bandages may be used to apply high levels of compression of about 40 to 50 mmHg on an ankle of average dimensions. Some compression bandages are capable of applying pressures in excess of 55 or 60 mmHg.

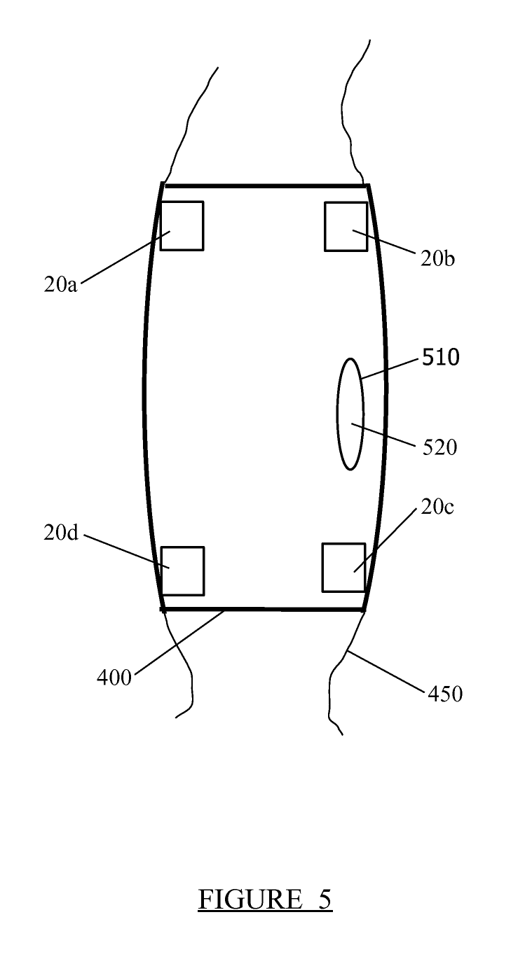

FIG. 5 shows a schematic side view of compressive sock 400, disposed on leg 450 of the user. Electrodes 20a-20d are disposed on leg 450, underneath compressive garment, sock or bandage 400. When the electrical stimulation unit is activated to contract at least one muscle on leg 450, a region of contraction 510 having a geometric center of contraction 520 is formed.

Preferably, compressive sock 400 covers, and delivers a compressive pressure to, the surface of at least a portion of region of contraction 510. As shown in FIG. 5, compressive sock 400 covers, and delivers a compressive pressure to, the surface of center of contraction 520.

The inventive device using electrical stimulation compounded by external pressure may be used on various limb segments on the body, including, but not limited to, the forearm, upper arm, and the foot, including the sole of the foot. Compression device 300 and compressive sock 400 may be specifically designed and configured to effectively envelope, and deliver pressure to, the surface of particular conical limb segments.

The compression hosiery, bandages, and the like used in accordance with the present invention advantageously deliver a compressive pressure of at least 5 mmHg, and more typically, at least about 8 mmHg.

The frequency, number, intensity and duration of muscle contractions may be heavily influenced by the nature of the signals passed to the electrodes.

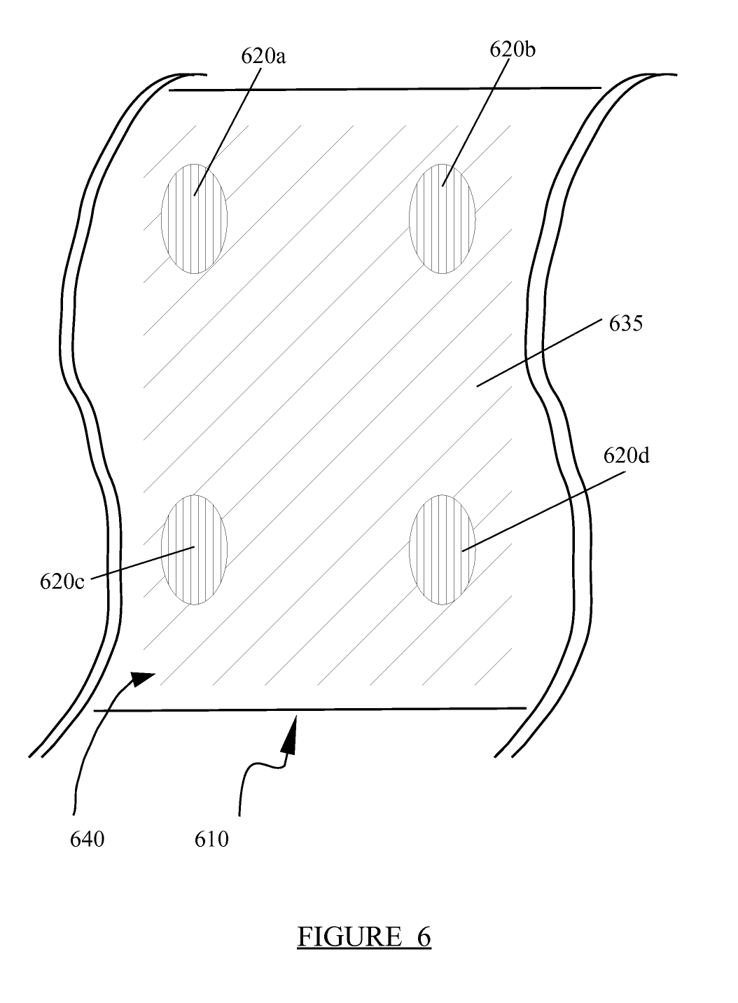

FIG. 6 shows a schematic perspective view of a portion of an inside or inner face 640 of a device of the present invention. Inside face 640 includes an inner face 635 of a flexible sheet 610 of at least one elastic compression garment or compression bandage. Attached to, and/or embedded in inside face 640 are surface electrodes 620a-620d.

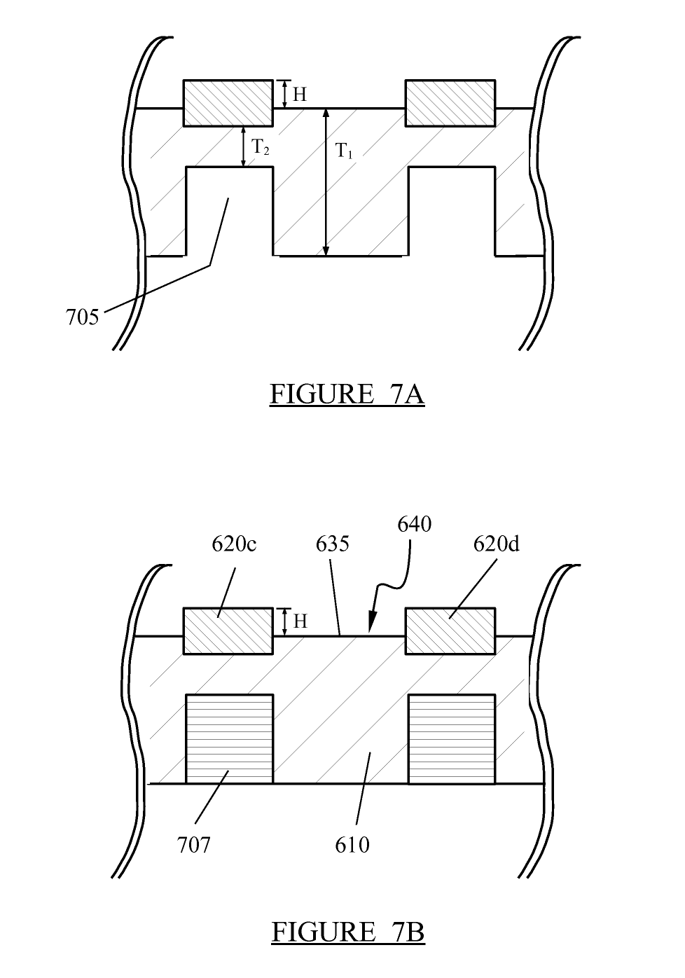

FIG. 7A is a schematic cross-sectional view of a portion of inside face 640 of FIG. 6. Inside face 640 includes inner face 635 of a flexible sheet 610 of the elastic compression garment or bandage. Attached to, and/or at least partially embedded within inside face 640 are the surface electrodes. In the particular cross-section of FIG. 7A, surface electrodes 620c and 620d are shown, partially embedded in flexible sheet 610.

We have found that the protrusion of surface electrodes 620a-620d away from the inside face may cause mild discomfort to a relatively healthy user, and even extreme discomfort to less healthy users, particularly those suffering from local ulcers, edemas, etc. Thus, it may be highly advantageous to recess the electrodes so that they are flush, or substantially flush with inner face 635 of flexible sheet 610. In the event that surface electrodes 620a-620d protrude from inner face 635 towards the surface of the limb segment, the protrusion height H may be preferably less than 0.6 mm, more preferably less than 0.4 mm, and most preferably less than 0.25 mm.

Alternatively or additionally, the thickness of flexible sheet 610 may be variable, whereby behind electrodes 620a-620d, the thickness T₂ of flexible sheet 610 is smaller than the thickness T₁ of flexible sheet 610 in an area surrounding electrodes 620a-620d (e.g., by having a recess or void volume 705 behind electrodes 620a-620d). Consequently, a second pressure exerted towards the limb segment via electrodes 620a-620d may be less than a first pressure exerted towards the limb segment in various areas of flexible sheet 610 surrounding electrodes 620a-620d. The first pressure may exceed the second pressure by at least 5 mm Hg, by at least 10 mmHg or by at least 15 mmHg.

Recess 705 may have a substantially larger surface area than the surface area of the electrode disposed therein. Consequently, the position of electrodes 620a-620d may be adjusted to suit the size and shape of leg segments of a wide variety of patients, such that the compression unit or garment may be of a universal nature.

The compression garment or bandage may be adapted whereby the superatmospheric pressure exerted by the electrodes may be less than 12 mmHg, less than 8 mmHg or less than 4 mmHg. This may be accomplished by means of recess or void volume 705. When multiple compressive bandage wraps are used, fewer wraps may be wrapped over electrodes 620a-620d to achieve this design criterion.

Typically, the thickness of the compression garment or bandage(s) above the electrodes may be less than the maximal thickness above the compression garment or bandage(s) by at least 2 mm, at least 1 mm, or at least 0.5 mm.

In another embodiment, provided in FIG. 7B in a schematic cross-sectional view, recess or void volume 705 of FIG. 7A is at least partially filled with a filler material 707, whereby, substantially as in the embodiment of FIG. 7A, the compressive forces or superatmospheric pressure acting on or through electrodes 620a-620d are reduced (typically by at least 5 mm Hg, by at least 10 mmHg or by at least 15 mmHg) with respect to the compressive forces or superatmospheric pressure delivered in various areas of flexible sheet 610 surrounding electrodes 620a-620d. Consequently, efficient, advantageous pressure may be exerted on the affected limb segment, without disadvantageously pressuring the tissue underneath the electrodes.

The localized increase in the flow of blood effected by the device and method of the present invention may be important for a wide variety of medical applications, including but not limited to alleviating CVI, inhibiting DVT, treating phlebitis, decreasing the amount of water retained, as in case of the lower limbs, improving blood and lymph circulation, thereby alleviating pain, and speeding up healing, particularly in the case of venous ulcers and the like. The restriction of blood flow by inducing the repeated contractual movement of muscular tissue against the natural flow of blood is also germane to a wide variety of medical applications, including various surgical procedures and edema reduction.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A non-invasive neuromuscular stimulation device for promoting a localized increase in a flow of blood through veins in a conical limb segment of a user by means of muscular contraction within the limb segmented compounded by mechanical compression, the device comprising:
(a) a compression unit, adapted to at least partially envelope the conical limb segment, and
(b) an electrical stimulation unit including:
(i) a plurality of electrodes including at least a first electrode and a second electrode, each of said first and second electrodes adapted to operatively contact the limb segment;
(ii) at least one signal generator, each said generator operatively connected to at least one of said electrodes, said at least one signal generator adapted to electrically connect to a power supply, whereby, when said plurality of electrodes are disposed on the limb segment, said at least one signal generator provides a series of electrical impulses to the limb segment via said plurality of electrodes to contract at least one skeletal muscle in the limb segment in at least one region of contraction, to effect the localized increase in the flow of blood through the veins, and
(iii) a control unit, associated with said signal generator, said control unit designed and configured to control said signal generator to produce said series of electrical stimulation impulses,
said electrodes physically attached to said compression unit and at least partially disposed thereunder,
wherein the conical limb segment is selected from the group consisting of an upper arm limb segment, a lower arm limb segment, an upper leg limb segment and a lower leg limb segment, said compression unit having an inside face adapted to deliver, to a surface of the limb segment, a superatmospheric pressure that is substantially constant over time, said pressure equaling at least 5 mmHg.

2. The device of claim 1, wherein said compression unit is selected from the group consisting of at least one compression bandage and at least one compression garment.

3. The device of claim 2, wherein said compression unit is said at least one compression garment.

4. The device of claim 2, wherein said compression unit is adapted to at least partially envelope a lower leg of the user.

5. The device of claim 3, wherein said inside face is adapted to contact the conical limb segment, said face having at least one receiving area adapted to receive said first and second electrodes.

6. The device of claim 5, wherein, when said compression garment is in a relaxed state, said first and second electrodes protrude from said inside face by less than 0.6 mm.

7. The device of claim 5, wherein, when said compression garment is in a relaxed state, said first and second electrodes are disposed within said garment, whereby a contact surface of said first and second electrodes is substantially flush with said inside face.

8. The device of claim 2, wherein said compression unit is an elastic compression unit.

9. The device of claim 1, wherein said compression unit is further adapted to envelope the limb segment to deliver said pressure to at least one region of contraction on the limb segment.

10. The device of claim 4, wherein said compression unit is further adapted, and disposed with respect to the electrodes, whereby said superatmospheric pressure is delivered to a calf of said lower leg, in a substantially identical location as a contraction of said skeletal muscle.

11. The device of claim 1, wherein said compression unit is further adapted to envelop substantially an entire circumference of the limb segment.

12. The device of claim 1, wherein said inside face has a first zone adapted to deliver a first pressure against a first portion of said surface of the limb segment and a second zone adapted to deliver a second pressure against the limb segment, said first pressure exceeding said second pressure.

13. The device of claim 12, said first pressure exceeding said second pressure by at least 5 mm Hg.

14. The device of claim 12, wherein at least one of said first and second electrodes is disposed within said second zone.

15. The device of claim 12, wherein an absolute pressure of said second pressure is less than 12 mmHg.

16. The device of claim 4, wherein said compression unit is adapted to at least partially envelope a gastrocnemius muscle of said lower leg of the user.

17. The device of claim 2, wherein said plurality of electrodes further includes at least a third electrode, the device further comprising:
(c) a switching mechanism designed and configured for switching electrical connections between said signal generator and at least each of said first, second, and third electrodes, according to a pre-determined sequence, to provide a first voltage differential between said first electrode and said second electrode, a second voltage differential between said second electrode and said third electrode, and a third voltage differential between said third electrode and another electrode of said plurality of electrodes.

18. The device of claim 5, wherein a thickness of said compression garment above said receiving area is less than a thickness of said compression garment outside of said receiving area.

19. The device of claim 12, wherein a first material of said compression garment, disposed above said first zone, is selected to exert a higher compressive force with respect to a second material of said compression garment, disposed above said second zone.

20. The device of claims 1, said compression unit adapted whereby said superatmospheric pressure is at least 8 mmHg, and whereby said superatmospheric pressure is at most 80 mmHg.
